(19) United States
(12) Patent Application Publication
(10) Pub. No.: US 2008/0187570 A1
(43) Pub. Date: Aug. 7, 2008

(54) Dna-Based Coatings For Implants

(75) Inventors: Johannes Arnoldus Jansen, Elst (NL); Roeland Johannes Maria Nolte, Nijmegen (NL); Nico Antoon Jaques Sommerdijk, Nuenen (NL); Xaverius Franciscus Walboomers, Nijmegen (NL); Jeroen Johannes Jacobus Paulus Van Den Beucken, Maasbree (NL); Matthijn Robert-Jan Vos, Amsterdam (NL)

Correspondence Address: FOLEY AND LARDNER LLP;SUITE 500, 3000 K STREET NW, WASHINGTON, DC, 20007, US

(73) Assignee: STICHTING VOOR DE TECHNISCHE WETENSCHAPPEN

(21) Appl. No.: 11/816,232

(22) PCT Filed: Feb. 14, 2006
(86) PCT No.: PCT/NL2006/050026

§ 371 Date: Jan. 16, 2008

(30) Foreign Application Priority Data
Feb. 15, 2005 (EP) 05101105.4

(51) Int. Cl. A61F 2/00; A61K 31/70; A61P 9/00

(52) U.S. Cl. 424/423; 514/44

(57) ABSTRACT

The invention relates to implants having a coating comprising DNA to improve the tissue-response at the site of implantation.

### FIELD OF THE INVENTION

The invention is in the field of implants having a coating to improve the tissue-response where the implant is implanted.

### BACKGROUND OF THE INVENTION

In implantology, a wide variety of materials is used to generate biomedical devices with satisfactory properties. Although bulk properties of materials largely determine their suitability for a given application, the biological response is mainly determined at the biomaterial surface, via the interactions with components of the biological surroundings. Modifications of the biomaterial surface have been a major research topic in the past decades. Many techniques have been developed to effectively modify biomaterial surfaces in order to improve the biological response. Examples include calcium phosphate deposition techniques for bone implants, and techniques by which recognition sites for biological and/or pharmaceutical compounds are introduced at biomaterial surfaces. The former techniques significantly increase the bioactivity of bone implants (Dorozhkin and Epple, Angew. Chem. Int. Ed. Engl. 2002, vol 41, 3130; Ducheyne and Qiu Biomaterials 1999, 20(23-24):2287), whereas the latter techniques improve (initial) attachment of cells, and provide a method to specifically deliver modulating compounds important in the biological response (e.g. cytokines and growth factors) at the site of implantation (Reyes and Garcia J. Biomed. Mat Res. 1999, vol 67A, 328; Bures et al. J. Conrol Release 2001 vol 72, 25).

In extending the concept of polyelectrolyte complexes between cationic polymers and DNA that have emerged as non-viral vectors for DNA (gene) delivery, WO 99/00071 describes stents that are coated with a polymer matrix in which DNA encoding a therapeutically useful protein is incorporated. It is demonstrated that a reporter enzyme in fact is expressed by artery cells, which means that the DNA must have entered the cells from the polymer matrix. As the DNA leaves the matrix and no longer covers the object onto which it is coated this approach is not suitable for implants that need to settle in the tissue into which they are implanted.

Also the use of DNA as a functional biomaterial, instead for its genetic information, has already been suggested (Yamada et al. Chemistry 2002, vol 8, 1407; Inoue et al. J. Biomed. Mat Res. 2003, vol 65A, 203), and pioneering efforts have resulted in the fabrication of DNA-containing bulk (bio)material, in particular self-standing, DNA-lipid films prepared by casting DNA-lipid complex (Fukushima et al. J. Dent. Res. 2001, vol 80, 1772), which demonstrated to cause no adverse reactions upon subcutaneous implantation in the backs of rats.

DE 10233099 discloses implants with a functionalized surface by providing a carbon containing layer on an implant and activating this layer through oxidation and/or reduction reactions and making it porous. The activated carbon is subsequently functionalized by adding amongst others DNA.

WO 02/47564 discloses implants having improved biocompatibility that are coated with amongst others DNA that is associated with a layer of metal hydride, viz. titanium hydride, zirconium hydride, tantalum hydride, hafnium hydride, niobium hydride, chromium hydride or vanadium hydride.

WO 03/072287 discloses methods of making implants by etching a microstructure onto the implant. Such an implant can be coated with DNA through an adhesive material such as gold. The DNA that is mentioned is used for its genetic information, viz. it is a gene encoding for nitric oxide or vascular endothelial growth factor.

The application of DNA as a coating material, however, is hampered by (a) its easy nucleolytic degradation, and (b) its solubility in aqueous solutions. The use of the previously mentioned DNA-containing bulk material resulted in easily detaching coatings on various materials.

Despite these drawbacks it is nevertheless an object of the present invention to provide implant objects with a coating comprising deoxyribonucleic acid (DNA) since this intriguing material will offer many advantages in implantology.

The present invention therefore seeks a solution to the problem of how to provide implant objects with coatings that comprise DNA, which coatings are stable, and remain stably attached to the implant object at least for as long as is necessary for the implant object to become sufficiently embedded in the tissue into which it is implanted and also are able to display the advantageous properties DNA has to offer to the tissue into which it is implanted.

### SUMMARY OF THE INVENTION

It was found that DNA can be successfully used as a coating on implants, not for its genetic properties but for the beneficial properties inherent to the structure of DNA. A coating comprising or consisting of DNA can be adsorbed onto an implant object, thereby resulting in an effectively DNA-coated implant object. In particular by incorporating DNA in a coating of an implant object by electrostatic interactions complexed with a multiple positively charged surface or layer, the DNA was stably attached to the implant object. Moreover the morphology of the DNA was retained, and thus able to exert the advantageous effects associated with the structure of DNA, while at the same time being protected from nucleolytic (e.g. serum nuclease) cleavage.

The invention thus relates to an implant object comprising a coating, said coating comprising a polynucleotide or equivalent thereof and wherein said coating is attached via adsorption to said implant object. In particular the invention relates to an implant object comprising a coating, said coating comprising a polynucleotide or equivalent thereof and a polycation. It is preferred the polynucleotide or equivalent thereof and polycation are in a double layer, or in other words the implant object is coated with a double layer of polycation and polynucleotide (equivalent). Such a double layer allows for the incorporation of biologically active components in the coating which subsequently exert their biological active function in a manner that is controlled by the coating. In particular the activity of the biologically active components can be controlled by the build-up of the polynucleotide (equivalent)—polycation coating, in particular in multiple-double layers and the modality of incorporation of biologically active components. By either incorporating biological active components to the surface of a double layer coating, or alternatively incorporating biological active components more deeply into a multilayered, or multiple double layer, coating, the release and effect of such biological active components can be influenced.

### DETAILED DESCRIPTION

In the context of this invention a polynucleotide or equivalent thereof refers to any polymer of nucleotides or equivalent building blocks representing nucleotides forming a polyanionic structure. Usually the negative charge is provided by or localized on a phosphate group (or equivalent) that links the nucleotides or nucleotide equivalent building blocks. In one embodiment the polynucleotide or equivalent thereof is selected from the group consisting of RNA, 2′-O-methyl RNA, or 2′-O-allyl RNA, DNA, morpholino polynucleotide, Peptide Nucleic Acid (PNA) and Locked Nucleic Acid (LNA).

In one embodiment the polynucleotide is DNA. The source of DNA is not critical. A suitable source of DNA is from the food industry where often DNA rich materials are discarded as industrial waste. Irrespective of its genetic information, the structural properties of DNA render this unique, natural material ideally suited for use as a coating for implants. The specific build-up of the DNA molecule ensures a versatile use at various implantation sites. The molecular structure of DNA in vertebrate species is homogeneous, and the non- or low-immunogenic properties of DNA (compared to other biological antigens like proteins and sugars) limits both innate and acquired immune responses. Additionally, DNA can incorporate other molecules via groove-binding and intercalation. This creates opportunities to specifically deliver desired biological mediators in the direct vicinity of the implantation site. Further, the high phosphate content in DNA beneficially affects, via the high affinity of phosphate for calcium ions, the deposition of calcium in the bone formation process.

In the context of this invention an implant object is any object that can be implanted in a human or animal body to help restoring damaged tissue structures, support or (partly) replace tissue and/or organs. Examples of implant objects are stents, fixation plates, fixation screws, medullary nails, acetabular cups, guided tissue regeneration membranes, oral implants, catheters, orthopaedic implants, pacemakers, heart valves, etc.

Adsorbed or adsorption in the context of this invention means any type of non-covalent attachment.

In one embodiment the coating comprising a polynucleotide or equivalent thereof is adsorbed onto an implant object via electrostatic interactions. One technique for electrostatically attaching a polynucleotide or equivalent thereof to an implant object is by electrostatic self-assembly (ESA). For example the surface of an implant can be treated to facilitate adsorption onto the implant like by the application of a gas plasma (glow discharge) alkaline or acid treatment (immersion of the substrate in a saturated sodium hydroxide or strongly acidic solution). Simply immersing the implant with a positively charged surface in a solution of for example DNA will result in self-assembly of the DNA onto the implant. Alternatively an implant may be thus treated to render the surface negatively charged. In such a case the implant can be immersed in a solution of a polycation which will self-assemble onto the implant. In a next step the implant can be immersed in a solution of DNA, resulting in an implant object that is coated with DNA that is electrostatically attached. Also in case of a polycation being electrostatically attached to an implant object and the polycation has the proper functionalisation, DNA or an equivalent thereof may be coupled to said polycation resulting in covalent interactions. Also the formation of multilayer films containing polynucleotide or equivalent thereof can be made by ESA, which is also known as layer by layer (LbL) assembly. Via this technique, polyelectrolyte multilayers (PEMs) can be generated through the alternated progressive adsorption of oppositely-charged polyelectrolytes via electrostatic interactions. Also at any point, apart from the first coating layer which (electrostatically) adsorbs onto the implant object, a further layer can be attached via other interactions than electrostatic, in particular via covalent attachment. In one embodiment the invention concerns an implant object comprising a coating, said coating comprising a polynucleotide or equivalent thereof and wherein said polynucleotide or equivalent thereof is attached via adsorption or in particular electrostatic interactions to said implant object.

Previously the ESA technique has been applied using DNA as a polyanionic building block. For example Pei et al., Biomacromolecules 2001, vol 2(2), 463, monitored the formation of multilayer films composed of poly(dimethyldiallylamonium chloride) (PDDA) and DNA continuously by real-time surface plasmon resonance, starting with a poly(ethyleneimine)-(PEI—) coated sensor chip. Also Luo et al, Biophys. Chem., 2001, vol 94(1-2), 11, studied multilayer films fabricated by LbL electrostatic deposition techniques of PDDA and DNA on glassy carbon electrodes and quartz slides.

Referring to the ESA technique the coated implant objects according to the invention can be prepared by subsequently immersing an implant in a solution of positively charged (poly)electrolyte and then in a solution of negatively charged polynucleotide or equivalent thereof, in particular DNA. This process can be repeated for a second time, or a third time or a fourth time or a fifth time or it can be even repeated 6, 7, 8, 9 or 10 times or even more, thereby creating a multilayer coating. Parameters that influence the formation and properties of such a multilayer coating are, besides the specific type of positively charged polyelectrolyte and even type of polynucleotide, concentration of the polyelectrolyte solutions, period of immersion, pH of the solutions, ionic strength of the solutions etc.

Furthermore, the method of fabrication (i.e. under immersion in aqueous solutions) shows that the polynucleotide containing PEMs are insoluble in water. Even immersion of PEMs in high ionic solutions (i.e. exceeding those in physiological conditions) does not cause dissociation of the PEM structure. Instead, changes in the ionic content of the polyelectrolyte solutions in the fabrication process are one of the means to modulate PEM properties, including layer thickness. It is well within the ambit of one of skill in the art to select those parameters resulting in a sufficiently coated implant object.

In one embodiment the positively charged (poly)electrolyte is a polycation and thus an implant object having a coating wherein a polynucleotide or equivalent thereof electrostatically interacts with a polycation in a double layer is an embodiment of the invention. Suitably the polycation is selected from the group consisting of Poly(Ala, Gly, Lys, Tyr) hydrobromide, Poly-D/L-arginine hydrochloride, Poly(Arg, Pro, Thr) hydrochloride, Poly(Arg, Trp) hydrochloride, Poly(Glu, Lys) hydrobromide, Poly(ethyleneimine), Poly-D/L-histidine, Poly-D/L-lysine, Polyvinylpyrrolidone, Poly(vinylpolypyrrolidone), Polyacrylamide, Poly(acrylamide-co-diallyldimethylammonium chloride), Poly(allylamine hydrochloride), Polyamilie (emeraldine salt), Poly[bis(2-chloroethyl)ether-alt-1,3-bis[3-(dimethylamino)propyl]urea]quarternized, Poly(diallyldimethylammonium chloride), Poly(4-vinylpyridinium tribromide. In a further embodiment the implant object comprises a coating comprising more than 1 double layer, e.g. 2, 3, 4, 5, 6, 7, 8, 9 or 10 or even more double layers, of polycation and polynucleotide.

In order to fully exploit the advantageous properties of DNA recited above, it is beneficial to control the morphology and the amount of DNA that is electrostatically immobilized in the coatings. To this end different types of multilayered DNA-coatings, which differ in the type of cationic polyelectrolyte used can be fabricated. The multilayered DNA-coatings can be characterized by UV-Vis spectrophotometry, atomic force microscopy (AFM), X-ray photospectroscopy (XPS), contact angle measurements, and Fourier transform infrared spectroscopy (FTIR). Also, the amount of DNA immobilized in the coatings can be analyzed using radiolabeled DNA.

The polynucleotide in the coating further may comprise functional additives such as drugs (antibiotics and/or anti-inflammation compounds) and/or signaling compounds, such as growth factors and/or cytokines and/or otherwise functional compounds that can be incorporated in a polynucleotide, in particular DNA. In short this means that an implant having a coating comprising DNA as defined above and that further comprises one or more biologically active components is a further embodiment of this invention.

The material of which the implant itself is made is dictated by the specific intended use of the particular implant. Particularly suited for materials for implants consist of or comprise metals, suitable metals for implants are niobium, tantalum, cobalt-chromium alloys, (stainless) steel and in particular titanium and titanium alloys. Several metals, such as for instance titanium, will have an oxide layer at the surface due their inherent properties and natural appearance. Other possible suitable materials to be coated are bioceramics, such as: aluminium oxide (alumina-ceramic; Al₂O₃), zirconium oxide (zirconia; ZrO2), calcium phosphate ceramic (CaP) and bioglass. Also implant objects may consist of or comprise polymeric materials such as polyethylene (PE), poly(ethyleneterephtalate) (PET), polytetrafluoroethylene (PFTE), polystyrene (PS), poly-L-lactic acid (PLLA), polydimethylsiloxane (PDMS), polyimide (PI), polyglycolic acid (PGA), polypropylene fumarate (PPF) or polybutylterephthalate (PBT). Further, composites of all above considered materials are candidate implant materials.

As already mentioned in the present invention it was found that DNA can be successfully used as a coating, not for its genetic properties but for the beneficial properties inherent to the structure of DNA. Therefore, in particular in a subject that is in need of receiving an implant, it is beneficial to use an implant object according to the present invention to improve tissue response where the implant is implanted. In particular the use of the present implant object is beneficial for healing of tissue surrounding the implant object and also the use of the present implant object is beneficial for support of biomineralization. In other words the invention concerns a method to improve tissue response where an implant is implanted, said method comprising implanting an implant object according to the present invention in a subject that is in need of receiving an implant. Also the method is for healing of tissue surrounding the implant object and also for support of biomineralization.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLE 1

The placement of implants into bodily tissues evokes responses directed against the devices, known as the foreign body response, and including inflammatory and wound healing processes. Surface properties largely determine the intensity and duration of these responses. Therefore, modulations at the surface of an implant material provide a means to control biological responses at the biomaterial interface. The novel approach in this invention is to use DNA as a coating material. Irrespective of its genetic information, DNA is a non- or low-immunogenic coating material with drug-delivery capacity in both soft and hard tissue environments. Accordingly DNA-coatings on implant objects using the Electrostatic Self-Assembly (ESA) technique were prepared and the cyto- and histocompatibility of multilayered DNA-coatings via in vitro assays with primary cells and an in vivo implantation study in rats were assessed.

### Materials & Methods:

Polyanionic DNA (+300 bp/molecule; sodium salt) was kindly provided by Nichiro Corporation (Yokosuka-shi, Kanagawa prefecture, Japan). Potential protein impurities in the DNA were checked using the BCA protein assay (Pierce, Rockford, Ill., USA) and measured to be below 0.20% w/w. Polycationic polyelectrolytes poly(allylamine hydrochloride) (PAH; MW 70000) and poly-D-lysine (PDL; MW 30000-70000) were purchased from Sigma (Sigma-Aldrich Chemie B. V., Zwijndrecht, the Netherlands).

Glass and titanium substrates were coated using the ESA-technique. Polycationic poly-D-lysine (PDL) or poly(allylamine hydrochloride) (PAH) and polyanionic DNA were alternately adsorbed as described below in order to obtain final coating architectures of either [PDL/DNA]₅ or [PAH/DNA]₅. Cytocompatibility was assessed using primary rat dermal fibroblasts (RDF) to monitor proliferation, cytotoxicity (MTT), and cell morphology (SEM). In vivo, specimens were implanted subcutaneously in the backs of rats for 4 and 12 weeks. Retrieved specimens (including surrounding tissues) were used for histological and histomorphometrical analyses to monitor tissue reactions.

### Generation of Multilayered DNA-Coatings

Multilayered DNA-coatings were generated using the ESA technique, as described by Luo et al., Biophys. Chem., 2001, vol 94(1-2), 11 with a few modifications. The cleaned substrates were immersed in an aqueous solution of either PDL (0.1 mg/ml) or PAH (1 mg/ml) for 30 minutes, allowing sufficient time for the adsorption of the first cationic polyelectrolyte layer onto the substrates. Subsequently, the substrates were washed in ultra-pure water (5 minutes, continuous water flow) and dried using a pressurized air stream. Thereafter, the substrates were alternately immersed in an anionic aqueous DNA solution (1 mg/ml) and the respective cationic polyelectrolyte solution for 7 minutes each, with intermediate washing in ultra-pure water (5 minutes, continuous water flow) and drying using a pressurized air stream. The build-up of the multilayered DNA-coatings was continued until a total of 5 double-layers was reached. These were designated either [PDL/DNA]₅ or [PAH/DNA]₅) (the denotation of coatings is restricted to indicating the number of double-layers, i.e. ½ represents only the cationic part of the double-layer).

### Coating Analysis—Atomic Force Microscopy (AFM)

The morphology of non-coated titanium-sputtered silicon, partial ([PDL/DNA]_{1/2} and [PAH/DNA]_{1/2}; [PDL/DNA]₂ and [PAH/DNA]₂; [PDL/DNA]_{31/2} and [PAH/DNA]_{31/2}) and complete ([PDL/DNA]₅ and [PAH/DNA]₅) multilayered DNA-coatings was analyzed using a Nanoscope IIIa AFM set-up (Digital Instruments, Buffalo, N.Y., USA).

Coating Analysis—Build-up of Multilayered DNA-Coatings with Radiolabeled DNA

The amount of DNA immobilized into the multilayered coatings was determined using radiolabeled DNA. Radiolabeled DNA was added to an aqueous DNA solution with a final concentration of 1 mg/ml. Concentrations of the aqueous solutions of the cationic polyelectrolytes were as described above. Multilayered coatings were fabricated as described above onto glass and titanium substrates. After the completion of 1, 2, 3, 4, and 5 double-layers, substrates were taken out of the fabrication process, immersed in scintillation fluid, and counted using a liquid scintillation counter. For comparison, samples containing 100 μl of the initial aqueous DNA solution (1 mg/ml) were counted. All experimental and control samples were present in 3-fold.

### Results

Using the ESA-technique, multilayered DNA-coatings were fabricated onto glass and titanium substrates. These multilayered DNA-coatings incorporated ˜3 μg DNA/cm²/double-layer and AFM demonstrated a nano-rough surface morphology.

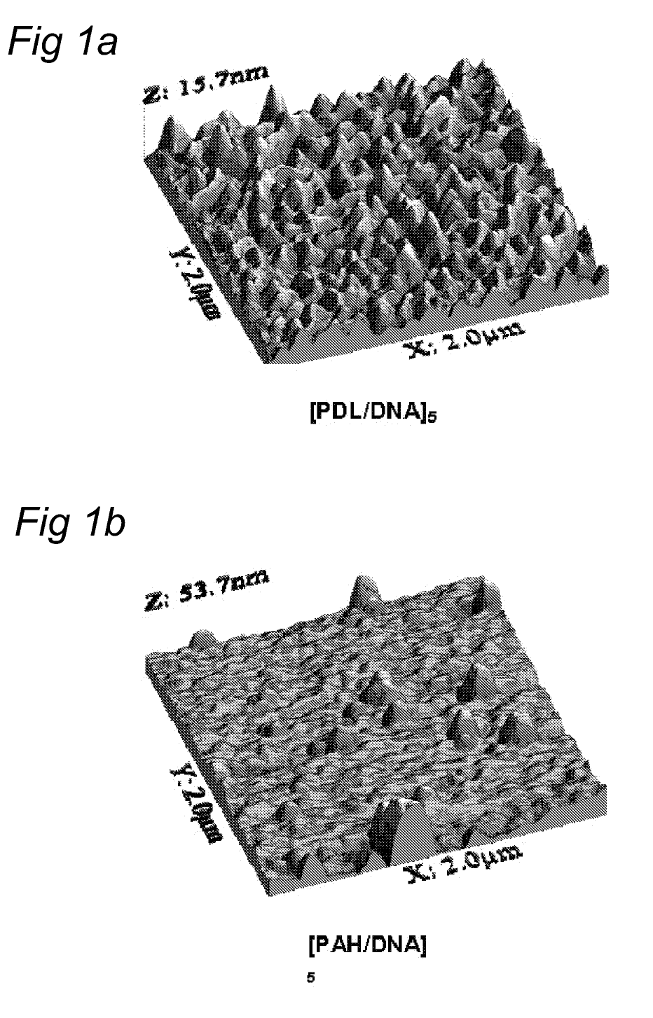

FIG. 1 shows three-dimensional reconstructions of AFM-images of (A) [PDL/DNA]₅ and (B) [PAH/DNA]₅ coatings.

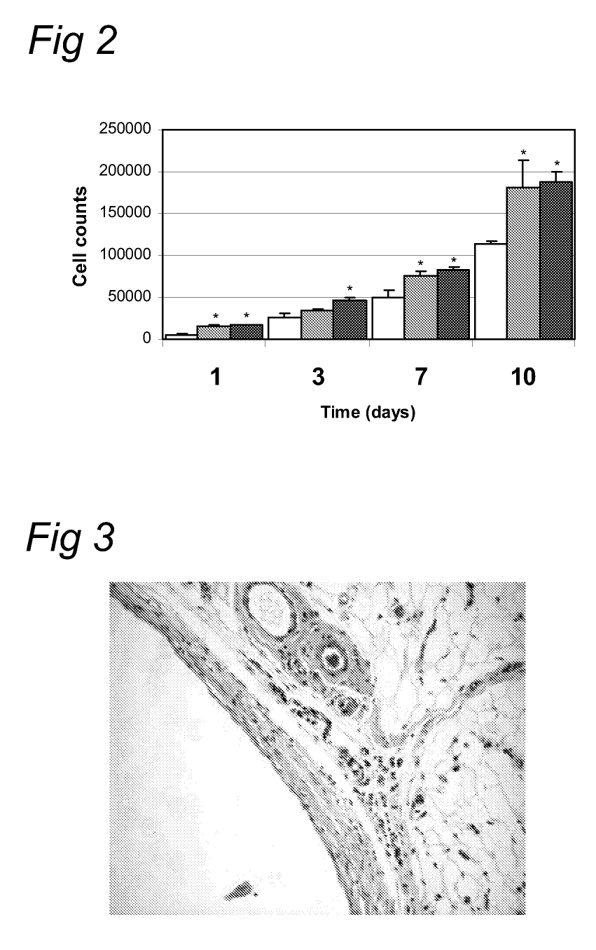

FIG. 2 shows proliferation of RDF cells on titanium substrates. White bars=non-coated controls, grey bars=[PDL/DNA]₅, black bars=[PAH/DNA]₅*Indicates significant difference compared to non-coated controls (p<0.05).

FIG. 3 shows a histological transversal section of the tissue capsule surrounding a glass [PDL/DNA]₅ substrate.

### Atomic Force Microscopy

The surface morphology of partial and complete multilayered DNA-coatings on titanium-sputtered silicon substrates was studied using AFM.

Analysis of heights of (partial and complete) multilayered DNA-coatings of both [PDL/DNA]- and [PAH/DNA]-double-layers shows for both types of coating, an optical increase in surface roughness. Further analysis of the surface roughness using RMS-values of both types of multilayered DNA-coatings showed significant increases with every step (i.e. 1½ double-layer) from [PDL/DNA]_{1/2} and [PAH/DNA]₂ during the build-up of the coatings. Furthermore, RMS-values of [PDL/DNA]-coatings were significantly lower than those of respective [PAH/DNA]-coatings after the deposition of 2 double-layers.

Typical differences in surface morphology between the two types of coatings included the spatial distribution of elevations, as well as their average height. Whereas [PDL/DNA]₅ showed a relatively homogenous morphological appearance with equally-spaced elevations of relatively low height (average 6 nm), [PAH/DNA]₅ showed randomly-distributed elevations of relatively large height (average 14 nm). To illustrate these differences, 3D-image reconstructions of both complete multilayered DNA-coatings are presented in FIG. 1.

### DNA Immobilization During the Build-Up of Multilayered Coatings

The amount of DNA (μg/substrate) immobilized into the multilayered DNA-coatings was analyzed using radiolabeled DNA. On glass the amount of DNA immobilized in the first double layer is higher for [PDL/DNA]-coatings than for [PAH/DNA]-coatings. An equal amount of DNA is immobilized into the first double-layer of both types of multilayered DNA-coatings on titanium substrates and both types of multilayered DNA-coatings immobilize a constant amount of DNA with each successively-adsorbed double-layer irrespective of the substrate material (i.e. glass or titanium). The amount of DNA immobilized into the multilayered DNA-coatings is approximately 1-15 μg DNA per cm² per double-layer.

In vitro experiments demonstrated increased proliferation of RDF cells on both types of multilayered DNA-coatings (p<0.05), irrespective of the underlying substrate material (FIG. 2). The MTT-assay revealed no cytotoxic effects of neither type of multilayered DNA-coating. Furthermore, no alterations in cell morphology were observed.

For the in vivo experiments, no signs of wound healing complications were observed during the implantation period. Histological analyses revealed no alterations in the tissue reactions to coated specimens compared to non-coated controls (FIG. 3). Fibrous capsule quality and the number of fibroblast layers in the fibrous tissue capsule showed no differences for coated specimens compared to controls.

### DISCUSSION AND CONCLUSIONS

To achieve optimal coverage of the oppositely-charged substrate one approach is to utilize a polyelectrolyte, which under neutral acidity is extremely charged (e.g. poly(ethyleneimine), PEI; or poly(styrenesulfonate), PSS) as the primary layer(s). This is however, not a necessity.

The growth of both [PAH/DNA]₅ and [PDL/DNA]₅ coatings was found to be linear. A substrate-dependent difference in the amount of DNA immobilized in the first double-layer can however occur as a consequence of the amount of cationic polyelectrolyte adsorbed in the primary adsorption layer and the inherent specific affinity of the used cationic polyelectrolytes for different types of substrate.

Multilayered DNA-coatings increase fibroblast proliferation, induce no cytotoxic effects, and do not alter fibroblast morphology. Upon implantation, multilayered DNA-coatings cause no differences in the capsule quality nor in the number of fibroblast layers in the capsule compared to non-coated controls. Therefore the cyto- and histocompatibility of multilayered DNA-coatings is demonstrated.

### EXAMPLE 2

In the field of implantology, control over tissue responses after the insertion of a biomaterial remains a challenge. Several approaches to modulate tissue responses have been investigated in the past decades, including topographical and/or chemical modulations at the biomaterial surface. Additionally, biomaterials have been combined with biologically active factors to induce desired tissue responses in the direct vicinity of the implant.

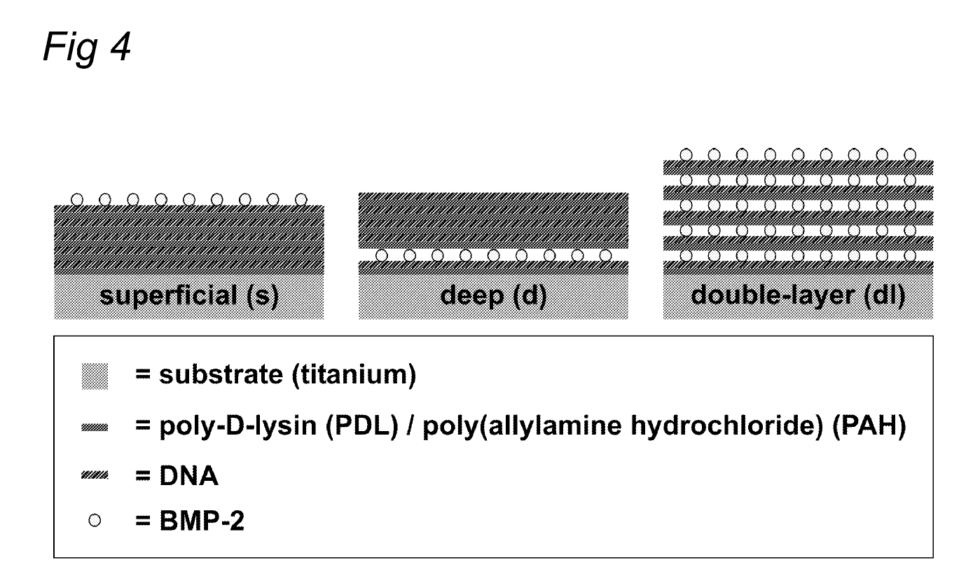

The current study focused on three types of functionalization of multilayered DNA-coatings (FIG. 4) built up from either poly-D-lysine (PDL) or poly(allylamine hydrochloride) (PAH) as the cationic component and DNA as the anionic component. The amounts of BMP-2 loaded into the multilayered DNA-coatings and its subsequent release characteristics were determined using radiolabeled BMP-2. Subsequently, the effect of BMP-2 functionalized multilayered DNA-coatings on the in vitro behavior of bone marrow-derived osteoblast-like cells was evaluated in terms of proliferation, differentiation, mineralization, and cell morphology.

FIG. 4 is a schematic representation of the different loading modalities of multilayered DNA-coatings with BMP-2.

### Materials & Methods:

Polyanionic DNA (300 bp/molecule; sodium salt) was kindly provided by the Central Research Laboratory of Nichiro Corporation (Kawasaki-shi, Kanagawa prefecture, Japan). Potential protein impurities in the DNA were checked using the BCA protein assay (Pierce, Rockford, Ill., US) and measured to be below 0.20% w/w (data not shown). Polycationic polyelectrolytes poly(allylamine hydrochloride) (PAH; MW ˜70000) and poly-D-lysine (PDL; MW 30,000-70,000) were purchased from Sigma (Sigma-Aldrich Chemie B. V., Zwijndrecht, the Netherlands). Recombinant human bone morphogenetic protein 2 (rhBMP-2; MW 32,000) was generously supplied by Yamanouchi Europe B. V. (Leiderdorp, the Netherlands). All materials were used without further purification.

### Substrate Preparation and Cleaning

Disc-shaped titanium substrates (diameter 12 mm; as machined) were used. Prior to the fabrication of multilayered DNA-coatings, substrates were cleaned ultrasonically in nitric acid (10% v/v), acetone, and isopropanol, respectively. Subsequently, the substrates were air-dried.

### Generation of Multilayered DNA-Coatings

Multilayered DNA-coatings were generated using the ESA-technique, as described previously. Briefly, the cleaned substrates were immersed in an aqueous solution of either PDL (0.1 mg/ml) or PAH (1 mg/ml) for 30 minutes, allowing sufficient time for the adsorption of the first cationic polyelectrolyte layer onto the substrates. Subsequently, the substrates were washed in ultra-pure water (5 minutes, continuous water flow) and dried using a pressurized air stream. Thereafter, the substrates were alternately immersed in an anionic aqueous DNA solution (1 mg/ml) and the respective cationic polyelectrolyte solution for 7 minutes each, with intermediate washing in ultra-pure water (5 minutes, continuous water flow) and drying using a pressurized air stream. The build-up of the multilayered DNA-coatings was continued until a total of 5 double-layers were reached. These coatings were designated either [PDL/DNA]₅ or [PAH/DNA]₅.

### Functionalization of Multilayered DNA-Coatings

Multilayered DNA-coatings were functionalized with rhBMP-2 according to three different loading modalities (FIG. 4). The loading modalities are designated superficial (s), deep (d), and double-layer (dl), depending on the location of the BMP-2. During the build-up of the multilayered DNA-coatings, rhBMP-2 was loaded (10 μl of a 10 μg/ml rhBMP-2 solution in 0.5% (w/v) BSA/PBS) at the appropriate location and allowed to adsorb for 7 minutes. Unless the rhBMP-2 was applied on top of the multilayered DNA-coatings, substrates were washed in ultra-pure water, after which the build up of the coatings was continued as described above. rhBMP-2 applied on top of the coatings (in case of s-loading and the final loading step in dl-loading) was allowed to dry at room temperature.

Radioiodination of rhBMP-2

rhBMP-2 was labeled with 1251 according to the iodogen method, as described previously. Briefly, to a 500 μl eppendorf vial containing 100 μg iodogen, 10 μl 0.5 M phosphate buffer (pH 7.4), 80 μl 50 mM phosphate buffer (pH 7.4), 10 μg rhBMP-2 (in 2.6 μl PBS), and 3 μl ¹²⁵I (0.3 mCi) were added. The vial was incubated at room temperature for 10 minutes. Subsequently, the quench reaction was initiated by adding 100 μl of a saturated Tyrosine solution in PBS. Finally, the reaction mixture was eluted with 0.5% BSA/PBS on a pre-rinsed disposable Sephadex G25M column (PD-10; Pharmacia, Uppsala, Sweden) to separate labeled rhBMP-2 from free ¹²⁵I. To prevent sticking of the rhBMP-2, pipette tips and vials used during the radioiodination procedure were silanized with SigmaCoat (Sigma).

The radiochemical purity of the ¹²⁵I-labeled rhBMP-2 was determined by instant thin-layer chromatography (ITLC) on Gelman ITLC-SG strips (Gelman Laboratories, Ann Arbor, Mich., USA) with 0.1 M citrate, pH 5.0 as the mobile phase. The radiochemical purity of the ¹²⁵I-labeled rhBMP-2 preparation was 97.3%, which indicates that 97.3% of the ¹²⁵I-label was covalently linked to rhBMP-2. The specific activity of the labeled rhBMP-2 was 14.1 μCi/μg.

Determination of rhBMP-2-Loading and in vitro rhBMP-2 Release

The amount of rhBMP-2 loaded in the functionalized multilayered DNA-coatings was determined using radiolabeled rhBMP-2. Functionalization was performed as described in the section ‘Functionalization of multilayered DNA-coatings’, with the exception that radiolabeled rhBMP-2 was used. For each type of coating, three substrates (n=3) were coated with a functionalized multilayered DNA-coating.

The loaded amount of rhBMP-2 was determined by measuring activity of the experimental substrates in a shielded well-type gamma counter (Wizard, Pharmacia-LKB, Sweden). The amount of gamma radiation from the deep (d-functionalization) and double-layer (dl-functionalization) loaded multilayered DNA-coatings was correlated to that of superficially loaded (s-functionalization) multilayered DNA-coatings, which was set at 100 ng.

To study the in vitro release characteristics of rhBMP-2, the substrates modified with a type of functionalized multilayered DNA-coating (n=3) were placed separately in 10 ml glass vials containing 4 ml PBS, and incubated statically at 37° C. for up to 8 weeks. At selected time points (4 hours, 1, 7, 14, 22, 28, 42, and 56 days) the samples were carefully transferred into new vials containing fresh PBS. Subsequently, the activity on the substrates was measured in a gamma counter. Standards were counted simultaneously to correct for radioactive decay.

In vitro Experiments

For cell culture experiments, 7 experimental groups were used, based on coating composition and loading modality:
1. [PDL/DNA]₅-s (superficial)
2. [PDL/DNA]₅-d (deep)
3. [PDL/DNA]₅-dl (double-layer)
4. [PAH/DNA]₅-s (superficial)
5. [PAH/DNA]₅-d (deep)
6. [PAH/DNA]₅-dl (double-layer)
7. control (non-coated titanium)

The use of a control consisting of non-coated titanium is justified as previous in vitro experiments have demonstrated that osteoblast-like cells behave similar on non-coated titanium compared to both [PDL/DNA]₅ and [PAH/DNA]₅ multilayered coatings with respect to cell proliferation, differentiation, mineralization, and morphology (unpublished data).

All substrates, coated with either type of multilayered DNA-coating or non-coated control substrates, were sterilized using a UW-irradiation treatment (254 nm; 4 hrs). The entire cell culture experiment was performed in two independent runs, using rat bone marrow cells from one rat per experimental run.

### Isolation and Pre-Culture of Rat Bone Marrow Cells

Rat bone marrow (RBM) cells were isolated an cultured according to the method adapted from Maniatopoulos et al. Briefly, the femora of male Wistar WU rats were retrieved, cleaned, and epiphyses were cut off. The marrow was flushed out of the remaining diaphyses using cell culture medium (α-MEM (Gibco) supplemented with 10% fetal calf serum (FCS; Gibco), 50 μg/ml ascorbic acid (Sigma), 10 mM Na-β-glycerophosphate (Sigma), 10⁻⁸ M dexamethasone (Sigma), and 50 μg/ml gentamycin (Gibco)). RBM cells of two femora were cultured under static conditions in cell culture medium in three 75 cm² culture flasks (Greiner Bio-One) for one day, after which the medium was refreshed to remove non-adherent cells. Subsequently, the attached cells were pre-cultured for another 6 days. After the primary culture of 7 days to obtain osteoblast-like cells, cells were detached using trypsin/EDTA (0.25% (w/v) trypsin, 0.02% (w/v) EDTA) and the total cell number was determined using a Coulter counter (Beckman Coulter Inc., Fullerton, Calif., USA). Finally, cells were seeded at a density of 1×10⁴ cells/cm² onto the experimental substrates, which were placed in a 24-wells plate (Greiner Bio-One). Cell culture medium was refreshed 1 day after cell seeding, and thereafter 3 times per week.

### Cell Proliferation

Cell proliferation curves were made based on a total cellular protein measurement. At 4, 8, 12, and 16 days post-seeding, the medium was removed and the cells were washed with PBS three times. Subsequently, the experimental substrates with attached cells were transferred into fresh 24-wells plates, and each experimental substrate was immersed in 1 ml ultra-pure water. These samples were frozen and thawed for 3 repetitive cycles, after which the cellular protein content in the aqueous samples was analyzed using a micro BCA protein assay (Pierce, Rockford, Ill., USA) according to the instructions of the manufacturer. In each experimental run, three samples per time point for each experimental condition (n=3) were used to ensure reproducibility.

### Alkaline Phosphatase Activity

The alkaline phosphatase (ALP) activity of the osteoblast-like cells was measured as a marker for early differentiation of osteoblast-like cells using the aqueous samples of the proliferation assay according to a previously described method. A volume of 80 μl of sample or standard and 20 μl of buffer solution (5 mM MgCl₂, 0.5 M 2-amino-2-methyl-1-propanol) was pipetted into a 96-wells plate (Greiner Bio-One) in duplo, and 100 μl of substrate solution (5 mM p-nitro-phenyl-phosphate) was added per well. Subsequently, the plate was incubated for one hour at 37° C., after which the reaction was stopped by adding 100 μl 0.3 M NaOH. Serial dilution of 4-nitrophenol (final concentrations 0-25 nM) were used for the standard curve. The plate was read in an ELISA reader at 405 nm. In each experimental run, three samples per time point for each experimental condition (n=3) were used.

### Calcium Deposition

The deposition of calcium was used as a marker of late differentiation of osteoblast-like cells. The amount of calcium deposited after 4, 8, 12, 16, and 24 days of cell culture was measured by the orthocresolphtalein complexone (OCPC) method (Sigma), as described previously. Briefly, the experimental substrates were washed twice using PBS, after which 1 ml 0.5 N acetic acid was added. After overnight incubation on a shaking apparatus, 300 μl working solution was added to 10 μl sample in a 96-wells plate (Greiner Bio-One). Working solution consisted of (a) OCPC solution (80 mg OCPC in 75 ml milliQ+0.5 ml 1 M KOH+0.5 ml 0.5 N acetic acid), (b) 14.8 M ethanolamine/boric acid buffer (pH=11), (c) 8-hydroxyquinoline (1 g in 20 ml 95% ethanol), and (d) milliQ, in a ratio of 5:5:2:88 (a:b:c:d). A standard curve was generated by preparing serial dilutions of CaCl₂ (0-100 μg/ml). For each experimental run, the calcium assay was performed using 3 substrates per experimental condition at each time point (n=3).

### Cell Morphology

Scanning electron microscopy (SEM) was performed to evaluate the morphological appearance of the cells. At 4 and 16 days post-seeding, substrates with attached cells were washed twice with PBS and fixed using gluteraldehyde (4% in 0.1 M cacodylate buffer) for 20 minutes. Subsequently, the substrates were washed twice with 0.1 M cacodylate buffer and dehydrated in a graded series of ethanol. Finally, the substrates were dried with tetramethylsilane, sputter coated with gold and examined using a JEOL 6310 SEM at an acceleration voltage of 10 kV.

### Statistical Analysis

Measurements were statistically evaluated with Graphpad® Instat 3.05 software (GraphPad Software Inc., San Diego, Calif., USA). Data of the in vitro release experiment and the cell culture experiments were analyzed using a one-way ANOVA, combined with a post-hoc Tukey-Kramer Multiple Comparisons test. The significance level was set at p<0.05.

### Results

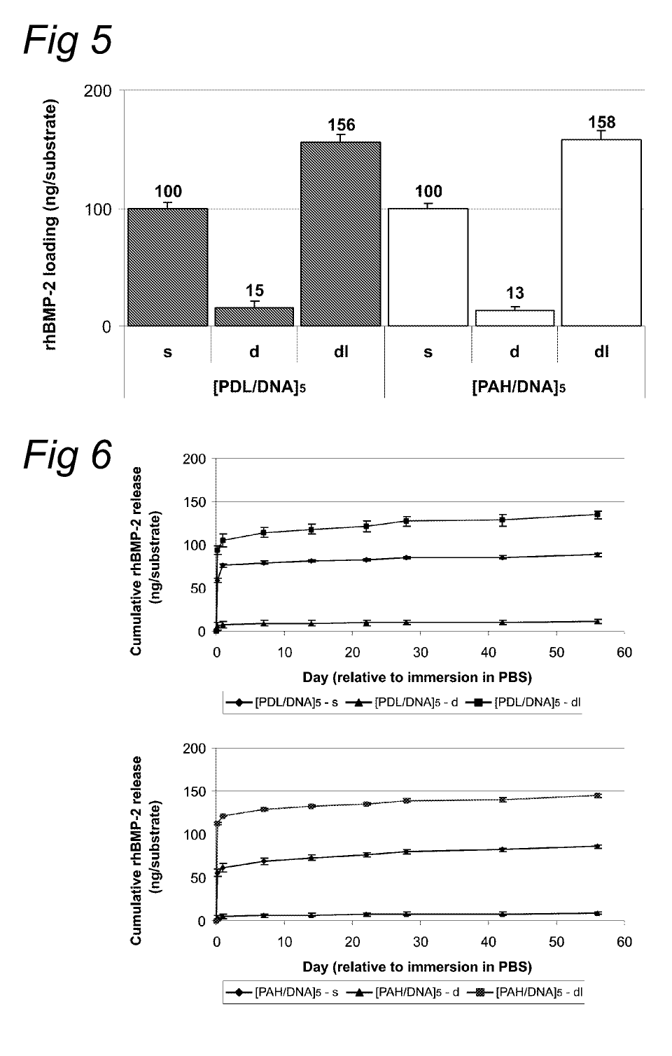

FIG. 5 shows the incorporation of rhBMP-2 into multilayered DNA-coatings. Results are presented as mean ±SD (n=3).

FIG. 6 shows in vitro release of rhBMP-2 from differently-loaded multilayered DNA-coatings after immersion in PBS. (A) Release characteristics of [PDL/DNA]-based multilayered DNA-coatings; (B) Release characteristics of [PAH/DNA]-based multilayered DNA-coatings (s=superficial, d=deep, dl=double-layer). Results are presented as mean ±SD (n=3).

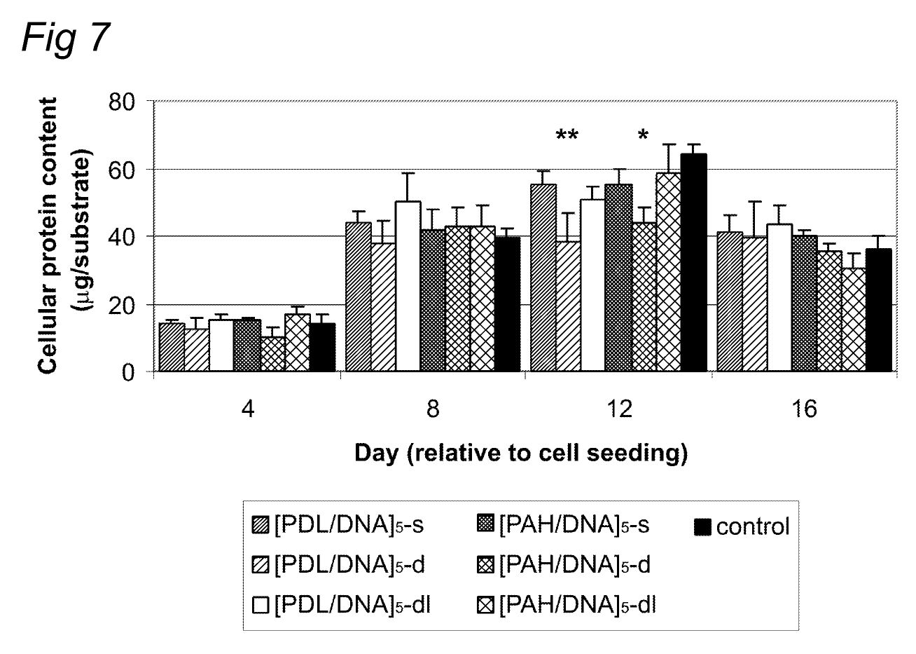

FIG. 7 shows proliferation of bone marrow-derived osteoblast-like cells on differently-loaded multilayered DNA-coatings. (*p<0.05; **p<0.01 compared to controls)

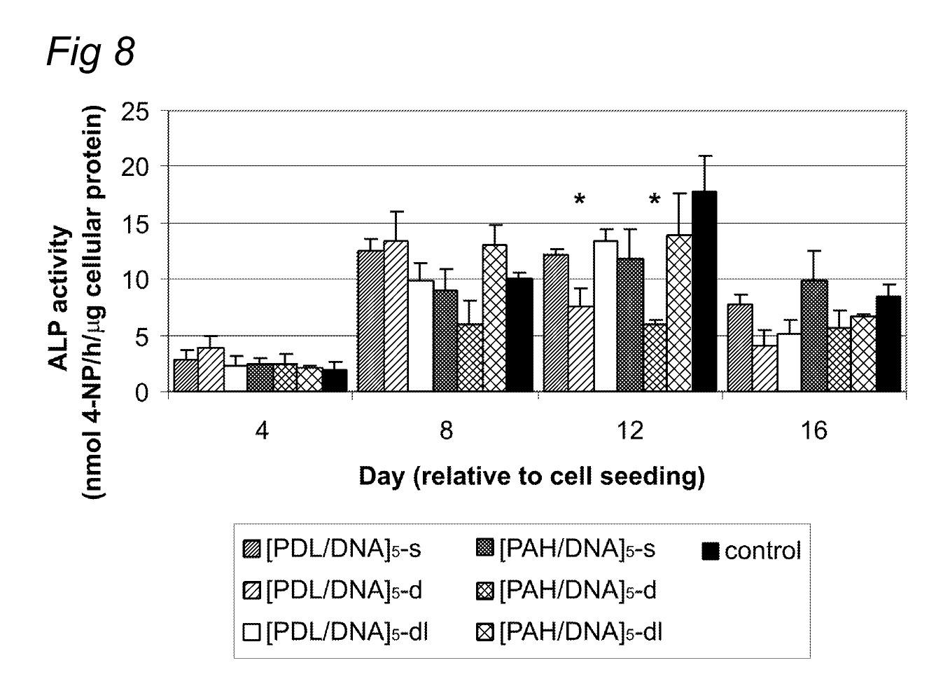

FIG. 8 shows alkaline phosphatase (ALP) activity of bone marrow-derived osteoblast-like cells on differently-loaded multilayered DNA-coatings. (*p<0.05 compared to controls)

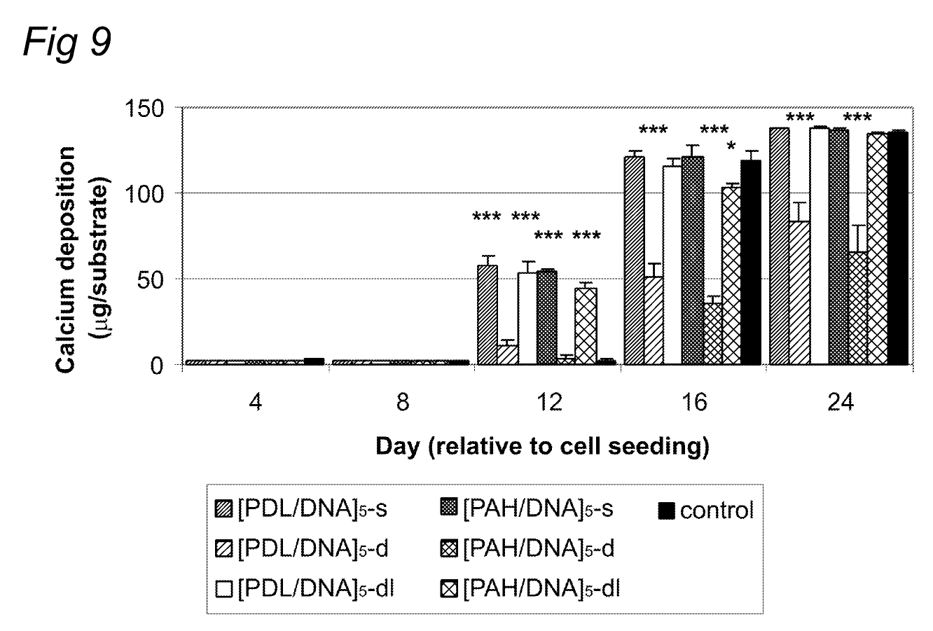

FIG. 9 shows mineralization (calcium deposition) by bone marrow-derived osteoblast-like cells on differently-loaded multilayered DNA-coatings. (*p<0.05; ***p<0.001 compared to controls)

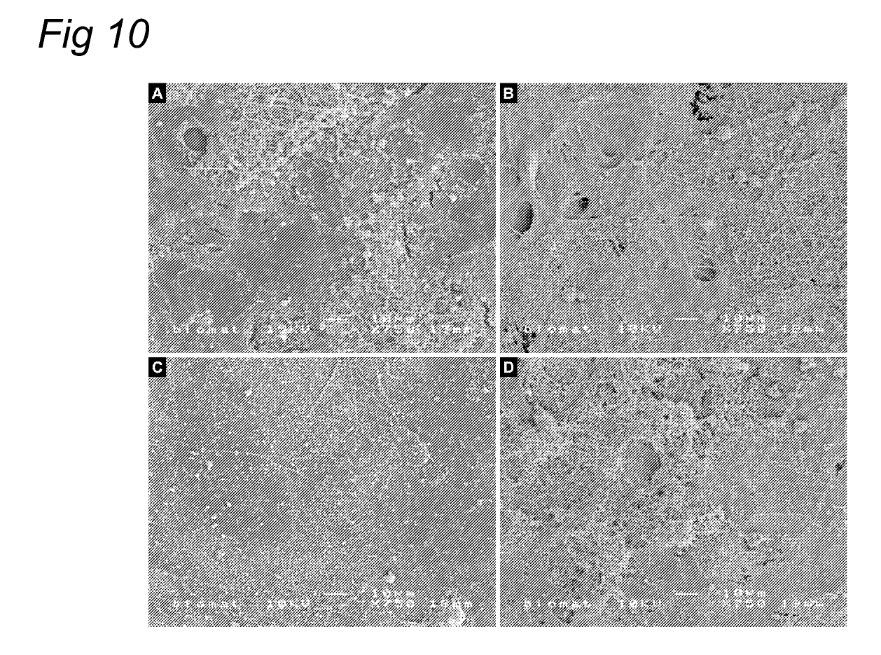

FIG. 10 shows scanning electron microscopy images of bone marrow-derived osteoblast-like cells after 16 days of culture on differently-loaded multilayered DNA-coatings. (A) non-coated control, (B) [PAH/DNA]₅-s, (C) [PAH/DNA]₅-d, and (D) [PAH/DNA]₅-dl.

Loading of Multilayered DNA-Coatings with rhBMP-2

The amounts of rhBMP-2 loaded into the multilayered DNA-coatings are presented in FIG. 5. The results demonstrate that the amount of rhBMP-2 incorporated into the differently-loaded multilayered DNA-coatings was highest with dl-loading, intermediate with s-loading, and lowest with d-loading (dl-loading>s-loading>d-loading). No statistically significant differences were observed between [PDL/DNA]-based coatings and [PAH/DNA]-based coatings.

In vitro Release of rhBMP-2 from Multilayered DNA-Coatings

The in vitro release characteristics of rhBMP-2 from multilayered DNA-coatings were determined using radiolabeled rhBMP-2. In FIGS. 6A and 6B, the cumulative release of rhBMP-2 out of the differently-loaded multilayered DNA-coatings is depicted. All differently-loaded multilayered DNA-coatings revealed an initial burst release within the first 24 hours of incubation in PBS, ranging from 35 to 75% of the initially loaded amount rhBMP-2. Proportionally, the burst release was low for d-loaded (47.6% for [PDL/DNA]-based and 34.8% for [PAH/DNA]-based multilayered DNA-coatings) and high for both s- and dl-loaded multilayered DNA-coatings (>60%). After the burst release, all differently-loaded multilayered DNA-coatings showed a sustained release, in which a continuous fraction of approximately 6-8% of the remaining rhBMP-2 was released in each week (Table 1). After an incubation period of 8 weeks, the cumulative rhBMP-2 release of the d-loaded multilayered DNA-coatings approximated 70%, whereas both the s-loaded and the dl-loaded DNA-coatings released approximately 85%, cumulatively. Statistical analysis revealed that actual cumulative amounts of released rhBMP-2 were highest for dl-loading, intermediate for s-loading, and lowest for d-loading. No statistically significant differences were observed between equivalently functionalized coatings based on either [PDL/DNA] or [PAH/DNA] regarding release characteristics in percentage terms (p>0.05).

TABLE 1
Release of rhBMP-2 from differently-loaded
multilayered DNA-coatings.

| Coating | Burst release (ng) | | Sustained release (ng/week) | |
|---|---|---|---|---|
| architecture | (0-24 h) | | (1-56 days | |
| [PDL/DNA]-s | 75.8 | (75.8%) | 1.6 | (6.7%) |
| [PDL/DNA]-d | 7.4 | (47.6%) | 0.5 | (5.8%) |
| [PDL/DNA]-dl | 105.1 | (65%) | 3.8 | (6.6%) |
| [PAH/DNA]-s | 61.3 | (61.3%) | 3.1 | (8.1%) |
| [PAH/DNA]-s | 4.7 | 34.8%) | 0.5 | (6.3%) |
| [PAH/DNA]-s | 121.1 | (73.7%) | 3.0 | (7.0%) |

Osteoblast-like Cell Behavior on rhBMP-2-Loaded Multilayered DNA-Coatings

The behavior of osteoblast-like cells on the differently-loaded multilayered DNA-coatings was evaluated to detect biological activity of the incorporated rhBMP-2.

### Cell Proliferation

The proliferation of osteoblast-like cells, based on total cellular protein content measurements, is depicted in FIG. 7. Osteoblast-like cells showed a similar proliferation pattern on all types of differently-loaded multilayered DNA-coatings and non-coated control substrates. After cell seeding, osteoblast-like cells started proliferating, reaching a maximum around day 12, after which a decrease was observed. Somewhat lower, but significantly different levels of cellular protein content were observed in both types of d-loaded multilayered DNA-coatings on day 12 ([PDL/DNA]₅-d vs. control, p<0.01; [PAH/DNA]₅-d vs. control, p<0.05). After 16 days of osteoblast-like cell culture, no significant different levels were observed between both types of d-loaded multilayered DNA-coatings and controls (p>0.05).

### Alkaline Phosphatase Activity

Osteoblast-like cells increased their ALP-activity on all experimental substrates during the first 12 days of culture, after which a rapid decrease in ALP-activity was observed (FIG. 8). Significant differences compared to controls (p<0.05) were observed on day 12 for both types of d-loaded multilayered DNA-coatings.

### Calcium Deposition

The deposition of a mineralized extracellular matrix by osteoblast-like cells was determined by measuring the amounts of calcium deposited on the experimental substrates during cell culture (FIG. 9). An accelerated calcium deposition by osteoblast-like cells was observed on s- and dl-loaded multilayered DNA-coatings compared to non-coated controls. On day 12, osteoblast-like cells on s- and dl-loaded multilayered DNA-coatings had deposited significantly increased amounts of calcium compared to non-coated controls (p<0.001). On the other hand, d-loaded multilayered DNA-coatings demonstrated to decrease calcium deposition by osteoblast-like cells. Significantly decreased amounts of deposited calcium were observed on these types of functionalized multilayered DNA-coatings on days 16 and 24 (p<0.001).

### Cell Morphology

The morphological appearance of the osteoblast-like cells cultured on the differently-loaded multilayered DNA-coatings was evaluated using scanning electron microscopy. At day 4, all differently-loaded multilayered DNA-coatings and non-coated controls were covered with a layer of osteoblast-like cells. No apparent differences in cell morphology were observed. In contrast, at day 16 d-loaded multilayered DNA-coatings showed an aberrant morphological appearance of osteoblast-like cells compared to all other experimental groups (FIG. 10). Many calcified globular accretions associated with collagen bundles were present on s-, and dl-loaded multilayered DNA-coatings, and non-coated controls. Less characteristics of mineralization were observed on d-loaded multilayered DNA-coatings.

### CONCLUSION

This study demonstrates the feasibility of multilayered DNA-coatings to be functionalized by embedding BMP-2 according to three different loading modalities: superficial (s), deep (d), and double-layer (dl). BMP-2 was incorporated proportionally into the multilayered DNA-coatings as: s+(4*d)=dl. The release profiles of all differently-loaded multilayered DNA-coatings showed an initial burst release, followed by a sustained release of the remaining BMP-2 for (at least) 8 weeks. In vitro culture experiments with rat bone marrow-derived osteoblast-like cells demonstrated that the loaded factor remained biologically active, as an accelerated calcium deposition was observed on s- and dl-loaded multilayered DNA-coatings, without affecting cell proliferation. In contrast, d-loaded multilayered DNA-coatings influenced the behavior of osteoblast-like cells by decreasing the deposition of calcium.

1-11. (canceled)

12. An object for implantation comprising a coating comprising a polynucleotide and a polycation.

13. The object according to claim 12 wherein the polynucleotide is a chemically modified polynucleotide.

14. The object according to claim 12, wherein said polynucleotide or equivalent thereof is selected from the group consisting of RNA, 2′-O-methyl RNA, 2′-O-allyl RNA, DNA, morpholino polynucleotide, Peptide Nucleic Acid (PNA) and Locked Nucleic Acid (LNA).

15. The object according to claim 14, wherein the polynucleotide is DNA.

16. The object according to claim 12, wherein the polynucleotide and the polycation are in a double layer.

17. The object according to claim 12 wherein the polycation is selected from the group consisting of Poly(Ala, Gly, Lys, Tyr) hydrobromide, Poly-D/L-arginine hydrochloride, Poly(Arg, Pro, Thr) hydrochloride, Poly(Arg, Trp) hydrochloride, Poly(Glu, Lys) hydrobromide, Poly(ethyleneimine), Poly-D/L-histidine, Poly-D/L-lysine, Polyvinylpyrrolidone, Poly(vinylpolypyrrolidone), Polyacrylamide, Poly(acrylannide-codiallyldimethylammonium chloride), Poly(allylamine hydrochloride), Polyamilie (emeraldine salt), Poly[bis(2-chloroethyl)ether-alt-1,3-bis[3-(dimethylamino)propyl]urea]quartemized, Poly(diallyldimethylammonium chloride), Poly(4-vinylpyridinium tribromide).

18. The object according to claim 16 comprising plurality of double layers of polycation and polynucleotide.

19. The object according to claim 12 further comprising a biologically active component.

20. The object according to claim 12, wherein the object is a stoat, a fixation plate, a fixation screw, a medullary nail, an acetabular cup, a guided tissue regeneration membrane, an oral implant, a catheter, an orthopaedic implant, a pacemaker, or a heart valve.

21. A method of improving tissue response at or near an implanted object comprising coating the object with a coating comprising a polynucleotide and a polycation.

22. The method according to claim 21 wherein the improvement is in healing of tissue at or near the implanted object.

23. The method according to claim 21 wherein the improvement is in biomineralization of tissue at or near the implanted object.
